(19) United States
(12) Patent Application Publication
(10) Pub. No.: US 2016/0220573 A1
(43) Pub. Date: Aug. 4, 2016

(54) COMBINATION THERAPIES FOR TREATING CANCERS

(71) Applicant: Gilead Sciences, Inc., Foster City, CA (US)

(72) Inventors: Julie A. Di Paolo, San Francisco, CA (US); Randall Mark Jones, Seattle, WA (US); Daniel B. Tumas, San Carlos, CA (US)

(21) Appl. No.: 15/010,906

(22) Filed: Jan. 29, 2016

Related U.S. Application Data
(60) Provisional application No. 62/111,604, filed on Feb. 3, 2015.

(51) Int. Cl. A61K 31/5377; A61K 31/495; A61K 31/496

(52) U.S. Cl. CPC A61K 31/5377; A61K 31/496; A61K 31/495

(57) ABSTRACT

Provided herein are methods that relate to a therapeutic strategy for treatment of cancer, including hematological malignancies. In particular, the methods include administration entospletinib and a Bcl-2 inhibitor, such as venetoclax, navitoclax, and ABT-737.

### CROSS REFERENCE TO RELATED APPLICATIONS

This application claims priority to and the benefit of U.S. Provisional Application No. 62/111,604, filed Feb. 3, 2015, which is hereby incorporated by reference in its entirety.

### FIELD

The present disclosure relates generally to therapeutics and compositions for treating cancers, and more specifically to the use of Spleen Tyrosine Kinase (Syk) inhibitors in combination with B-cell CLL/lymphoma 2 (Bcl-2) inhibitors for treating cancers.

### BACKGROUND

Syk inhibitors useful as anticancer agents include entospletinib, discussed in Phase 2 Trial of Entospletinib (GS-9973), a Selective SYK Inhibitor, in Follicular Lymphoma (FL), Sharman et al., Blood, 124(21), Dec. 6, 2014.

Various compounds that inhibit the activity of anti-apoptotic Bcl proteins are known in the art. Several Bcl-2-selective apoptosis inducing compounds may be used in treating cancer. However, some Bcl-2 inhibitors may cause thrombocytopenia and have limited use in clinical treatments (see e.g., Zhang et al., Cell Death and Differentiation 14: 943-951, 2007). Thus, there remains a need for alternative therapies to treat cancer in humans.

### BRIEF SUMMARY

Provided herein are methods for treating cancer that involve the administration of a Syk inhibitor in combination with a Bcl-2 inhibitor. In some aspects, provided is a method for treating cancer in a human in need thereof, comprising administering to the human a therapeutically effective amount of a Syk inhibitor and a therapeutically effective amount of a Bcl-2 inhibitor.

In some embodiments, the Syk inhibitor is 6-(1H-indazol-6-yl)-N-(4-morpholinophenyl)imidazo[1,2-a]pyrazin-8-amine, or a pharmaceutically acceptable salt or hydrate thereof. In some variations, the Syk inhibitor is a mesylate salt of 6-(1H-indazol-6-yl)-N-(4-morpholinophenyl)imidazo[1,2-a]pyrazin-8-amine, or a hydrate thereof. Examples of mesylate salts and formulations thereof useful in the present methods may be seen in U.S. 2015/0038504 (Casteel et al.) and U.S. 2015/0038505 (Elford et al.).

In some embodiments, the Bcl-2 inhibitor is:
(4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide);
4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide; or
4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl) sulfonyl)benzamide;
   or a pharmaceutically acceptable salt thereof.

Provided herein are also articles of manufacture and kits that comprise the Syk inhibitor and the Bcl-2 inhibitors described herein.

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein is a method for treating cancer in a human in need thereof, comprising administering to the human a therapeutically effective amount of a Syk inhibitor and a therapeutically effective amount of a Bcl-2 inhibitor. Provided are also compositions (including pharmaceutical compositions, formulations, or unit dosages), articles of manufacture and kits comprising a Syk inhibitor and a Bcl-2 inhibitor.

### Compounds

In some variations, the Syk inhibitor is Compound A1, or a pharmaceutically acceptable salt or hydrate thereof. Compound A1 has the structure:

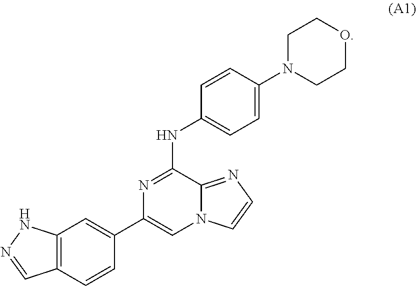 [SMILES: c1cn2cc(-c3ccc4cn[nH]c4c3)nc(Nc3ccc(N4CCOCC4)cc3)c2n1]

In some variations, the Syk inhibitor is a mesylate salt of Compound A1, or a hydrate thereof. In one variation, the mesylate salt of Compound A1 may be a mono-mesylate salt or a bis-mesylate salt. In another variation, the Syk inhibitor is a monohydrate, bis-mesylate salt of Compound A1. Compound A1 may be synthesized according to the methods described in U.S. Pat. No. 8,450,321. Compound A1 may be referred to as 6-(1H-indazol-6-yl)-N-(4-morpholinophenyl)imidazo[1,2-a]pyrazin-8-amine or entospletinib.

In particular embodiments the compound of Formula IA

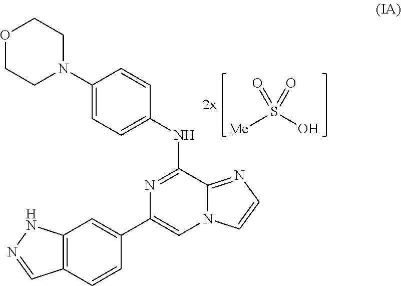

a crystalline form of the bis-mesylate (MSA) salt, is utilized. In some variations, the bismesylate salt is of Polymorph Form 3 described in U.S. 2015/0038504 (Casteel et al.) and U.S. 2015/0038505 (Elford et al.). In some variations, Polymorph Form 3 is used, which has an X-ray diffraction (XRPD) pattern comprising 20-reflections (±0.2 degrees): 13.8, 16.9, 22.9, and 26.1. In some embodiments, polymorph Form 3 has an X-ray diffraction (XRPD) pattern comprising at least one or more; at least two or more; or at least 3 or more of the 20-reflections (±0.2 degrees): 13.8, 16.9, 22.9, and 26.1. In some variations, polymorph Form 7, as described by Casteel et al. and Elford et al., is used, which has an X-ray diffraction (XRPD) pattern comprising 20-reflections (±0.2 degrees): 4.9, 9.8, and 26.7. In some embodiments polymorph Form 7 has an X-ray diffraction (XRPD) pattern comprising at least one or more; or at least two or more of the 20-reflections (±0.2 degrees): 4.9, 9.8, and 26.7.

The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (melting point).

For example, in one embodiment, the polymorph Form 3 of bis-mesylate salt (IA) used as described herein is substantially crystalline. In another embodiment, Form 7 of bis-mesylate salt (IA) used as described herein is substantially crystalline. In some embodiments, a compound that is substantially crystalline has greater than 50%; or greater than 55%; or greater than 60%; or greater than 65%; or greater than 70%; or greater than 75%; or greater than 80%; or greater than 85%; or greater than 90%; or greater than 95%, or greater than 99% of the compound present in a composition in crystalline form. In other embodiments, a compound that is substantially crystalline has no more than about 20%, or no more than about 10%, or no more than about 5%, or no more than about 2% in the amorphous form.

In some variations, the Bcl-2 inhibitor is Compound B1, Compound B2, or Compound B3, or a pharmaceutically acceptable salt thereof.

Compound B1 has the structure:

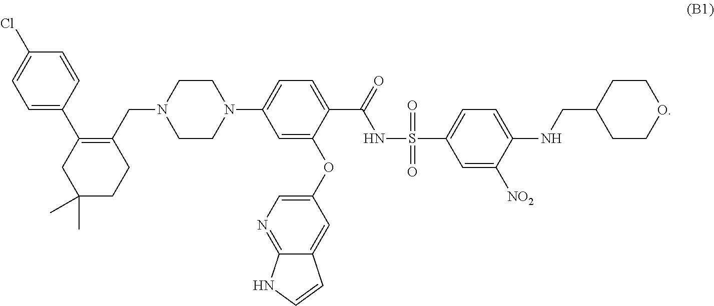 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Compound B2 has the structure:

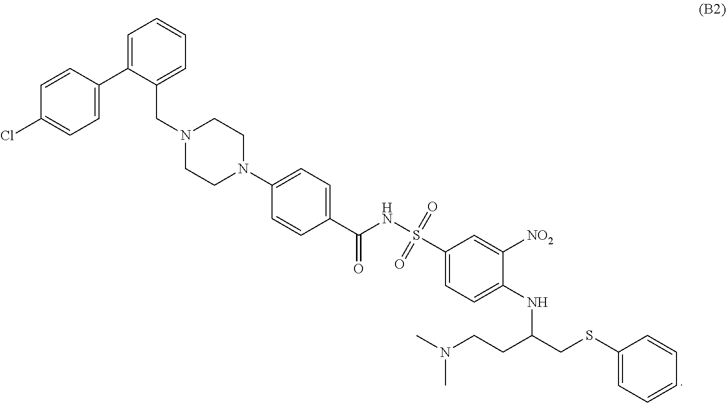 [SMILES: CN(C)CCC(CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

Compound B3 has the structure:

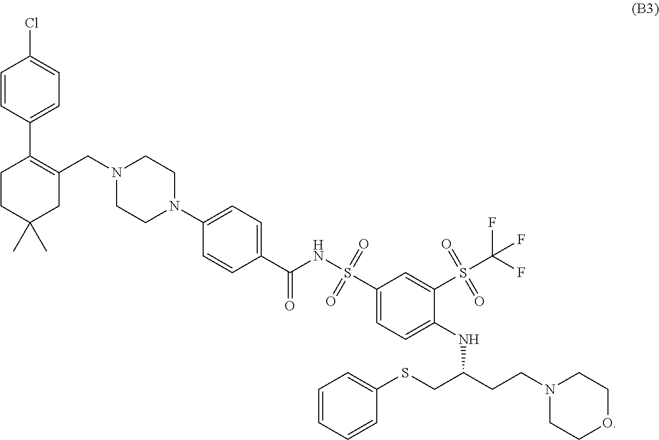 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, Compound B1, or a pharmaceutically acceptable salt thereof, is used in combination with Compound A1, or a pharmaceutically acceptable salt or hydrate thereof. In other embodiments, Compound B2, or a pharmaceutically acceptable salt thereof, is used in combination with Compound A1, or a pharmaceutically acceptable salt or hydrate thereof. In yet other embodiments, Compound B3, or a pharmaceutically acceptable salt thereof, is used in combination with Compound A1, or a pharmaceutically acceptable salt or hydrate thereof.

Compounds B1, B2 and B3 are commercially available, and their methods of synthesis are generally known in the art. For example, Compounds B1, B2 and B3 may be synthesized according to U.S. Patent Application Publication Nos. 2010/0305122, 2007/0072860, or 2007/0027135.

In addition to the chemical structure, Compound B1 may also be referred to or identified as (4-(4-{[2-(4-chlorophenyl)-4,4-dimethylcyclohex-1-en-1-yl]methyl}piperazin-1-yl)-N-({3-nitro-4-[tetrahydro-2H-pyran-4-yl-methyl)amino]phenyl}sulfonyl)-2-(1H-pyrrolo[2,3-b]pyridin-5-yl-oxy)benzamide), 4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)-benzamide, ABT-199 GDC 0199, or Venetoclax. Crystalline forms of Compound B1 useful in the methods and combinations herein can be seen in WO 2012/071336 (Catron et al.).

In some variations herein, Compound B1 is utilized in forms disclosed in WO 2012/071336 (Catron et al.). In one embodiment the crystalline form is Compound B1 free base anhydrate, characterized by a powder X-ray diffraction pattern having at least one peak selected from those at 6.3, 7.1, 9.0, 9.5, 12.5, 14.5, 14.7, 15.9, 16.9, and 18.9 degrees 28 (pattern A in WO 2012/071336), with each peak being ±0.2 degrees 28, when measured at about 25° C. with Cu Kα radiation at 1.54178 Å.

In another embodiment, the crystalline form is Compound B1 free base anhydrate, characterized by a powder X-ray diffraction pattern having at least one peak selected from those at 5.8, 7.7, 8.3, 9.9, 13.0, 13.3, 14.2, 15.3, 16.6, 17.9, 18.3, 19.8, 20.7, 21.2, 21.9, 22.5, 23.6, and 24.1 degrees 28 (pattern B in WO 2012/071336), each peak being ±0.2 degrees 28, when measured at about 25° C. with Cu Kα radiation at 1.54178 Å.

In another embodiment, the crystalline form is Compound B1 free base hydrate, characterized by a powder X-ray diffraction pattern having at least one peak selected from those at 5.8, 7.6, 7.9, 10.7, 11.7, 14.0, 15.3, 15.8, 17.4, 18.3, 19.9, 20.4, 20.7, 22.5, 24.9, 25.8, and 26.7 degrees 28 (pattern C in WO 2012/071336), each peak being ±0.2 degrees 28, when measured at about 25° C. with Cu Kα radiation at 1.54178 Å.

In another embodiment, the crystalline form utilized is Compound B1 free base hydrate, characterized by a powder X-ray diffraction pattern having at least one peak selected from those at 3.3, 6.4, 7.1, 7.3, 10.1, 11.4, 13.2, 14.4, 14.6, 15.1, 15.8, 16.2, 17.2, 17.6, 18.0, 18.6, 19.0, 19.5, 19.8, 20.2, 20.7, 21.0, 22.5, 23.0, 26.0, 28.9, and 29.2 degrees 28 (pattern D in WO 2012/071336), each peak being ±0.2 degrees 28, when measured at about 25° C. with Cu Kα radiation at 1.54178 Å.

In a further embodiment, the crystalline form is Compound B1 free base dichloromethane solvate, characterized by a powder X-ray diffraction pattern having at least one peak selected from those at 5.9, 7.1, 9.6, 10.0, 10.7, 11.1, 13.2, 14.8, and 18.2 degrees 28, each peak being ±0.2 degrees 28 (pattern E in WO 2012/071336), when measured at about 25° C. with Cu Kα radiation at 1.54178 Å.

In an additional embodiment, the crystalline form is Compound B1 free base dichloromethane solvate, characterized by a monoclinic lattice type and P21/n space group having unit cell lengths for the three axes of about (a) 13.873 Å, (b) 12.349 Å, (c) 29.996 Å and the three unit cell angles of about (α) 90.00°, (β) 92.259°, and (γ) 90.00°, as described in WO 2012/071336.

In a different embodiment the crystalline form is Compound B1 free base ethyl acetate solvate, characterized by a powder X-ray diffraction pattern having at least one peak selected from those at 5.8, 7.1, 9.5, 9.9, 10.6, 11.6, 13.1, 13.8, 14.8, 16.0, 17.9, 20.2, 21.2, 23.2, 24.4, and 26.4 degrees 28 (pattern F in WO 2012/071336), each peak being ±0.2 degrees 28, when measured at about 25° C. with Cu Kα radiation at 1.54178 Å.

In a different embodiment, the crystalline form is Compound B1 free base ethyl acetate solvate, characterized by a powder X-ray diffraction pattern having at least one peak selected from those at 3.3, 6.5, 7.0, 7.3, 9.2, 9.7, 11.2, 11.4, 11.9, 12.9, 14.4, 14.9, 15.8, 16.2, 17.2, 17.4, 17.8, 18.5, 18.9, 19.4, 20.1, 20.7, 20.9, 22.0, 22.7, 23.4, 23.8, 24.7, 25.9, 27.0, and 28.9 degrees 28 (pattern Gin WO 2012/071336), each peak being ±0.2 degrees 28, when measured at about 25° C. with radiation at 1.54178 Å.

In a separate embodiment, the crystalline form is Compound B1 free base acetonitrile solvate, characterized by a powder X-ray diffraction pattern having at least one peak selected from those at 5.8, 7.4, 7.6, 10.2, 13.0, 13.6, 14.9, 16.4, 17.0, 17.5, 18.2, 19.4, 19.7, 20.4, 21.0, 21.2, 21.8, 22.4, 22.9, 24.2, 24.3, 26.1, and 29.2 degrees 28 (pattern H in WO 2012/071336), each peak being ±0.2 degrees 28, when measured at about 25° C. with radiation at 1.54178 Å.

In another embodiment the crystalline form is Compound B1 free base acetonitrile solvate, characterized by a triclinic lattice type and PI space group having unit cell lengths for the three axes of about (a) 12.836 Å, (b) 13.144 Å, (c) 15.411 Å and the three unit cell angles of about (α) 92.746°, (β) 95.941°, and (γ) 113.833°, as described in WO 2012/071336.

In an additional embodiment, the crystalline form is Compound B1 free base acetonitrile solvate, characterized by a powder X-ray diffraction pattern having at least one peak selected from those at 6.4, 6.9, 7.7, 8.8, 9.4, 11.1, 12.3, 12.8, 16.5, 17.0, 17.4, 18.3, 18.6, 19.0, 19.2, 20.3, 21.6, 22.3, 22.9, and 23.7 degrees 28 (pattern I in WO 2012/071336), each peak being ±0.2 degrees 28, when measured at about 25° C. with radiation at 1.54178 Å.

In a separate embodiment, the crystalline form is Compound B1 free base acetone solvate, characterized by a powder X-ray diffraction pattern having at least one peak selected from those at 6.0, 6.8, 8.0, 9.0, 9.7, 11.2, 11.9, 12.6, 14.7, 15.0, 15.2, 15.8 16.4, 16.6, 17.6, 17.8, 17.9, 18.7, 20.2, 20.8, 21.6, 22.2, 22.6, 23.3, 23.8, 24.0, 24.4, 26.8, 27.1, 28.0, and 28.2 degrees 28 (pattern J in WO 2012/071336), each peak being ±0.2 degrees 28, when measured at about 25° C. with radiation at 1.54178 Å.

In another embodiment, the crystalline form is Compound B1 hydrochloride, characterized by a powder X-ray diffraction pattern having at least one peak selected from those at 5.1, 5.9, 7.7, 9.9, 10.2, 10.8, 13.6, 14.0, 15.4, 15.9, 16.2, 17.6, 18.3, 18.7, 19.7, 19.9, 20.1, 20.4, 20.7, 20.9, 22.9, and 26.2 degrees 28 (Pattern K in WO 2012/071336), each peak being ±0.2 degrees 28, when measured at about 25° C. with Cu Ka radiation at 1.54178 Å.

In a different embodiment the crystalline form is Compound B1 free base hydrochloride, characterized by a triclinic lattice type and P1 space group having unit cell lengths for the three axes of about (a) 10.804 Å, (b) 12.372 Å, (c) 19.333 Å and the three unit cell angles of about (α) 76.540°, (β) 87.159°, and (γ) 70.074°, as described in WO 2012/071336.

In still another embodiment, the crystalline form is Compound B1 free base hydrochloride hydrate, characterized by a powder X-ray diffraction pattern having at least one peak selected from those at 4.6, 8.7, 9.6, 9.9, 12.3, 14.9, 15.7, 17.6, 18.1, 18.4, 19.3, 19.6, 21.0, 23.3, 23.9, 24.8, 26.5, 27.2, 27.4, 29.0, and 30.1 degrees 28 (pattern L in WO 2012/071336), each peak being ±0.2 degrees 28, when measured at about 25° C. with Cu Ka radiation at 1.54178 Å.

In a different embodiment, the crystalline form is Compound B1 free base sulfate, characterized by a powder X-ray diffraction pattern having at least one peak selected from those at 4.8, 7.7, 8.3, 9.7, 10.2, 12.0, 12.6, 14.5, 15.4, 17.4, 17.9, 18.4, 19.1, 19.5, 21.0, 22.4, 23.3, 23.9, 25.1, and 26.8 degrees 28 (pattern M in WO 2012/071336), each peak being ±0.2 degrees 28, when measured at about 25° C. with Cu Ka radiation at 1.54178 Å.

In another embodiment, the crystalline form is Compound B1 free base tetrahydrofuran, characterized by a powder X-ray diffraction pattern having a least one peak selected from those at 4.0, 4.6, 8.0, 8.5, 9.4, 14.6, 17.1, 17.4, 17.8, 18.1, 19.2, 19.5, 20.1, 20.4, 20.5, and 21.7 degrees 28 (pattern N in WO 2012/071336), each peak being ±0.2 degrees 28, when measured at about 25° C. with Cu Ka radiation at 1.54178 Å.

Compound B2 may be referred to or identified as 4-(4-((4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide; 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-benzamide; or ABT-737.

Compound B3 may be referred to or identified as (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide; 4-(4-{[2-(4-chlorophenyl)-5,5-dimethyl-1-cyclohexen-1-yl]methyl}-1-piperazinyl)-N-[(4-{[(2R)-4-(4-morpholinyl)-1-(phenylsulfanyl)-2-butanyl]amino}-3-[trifluoromethyl)sulfonyl]phenyl)sulfonyl]benzamide, Navitoclax, or ABT-263.

In one embodiment, Compound B3 is used as the ABT-263 bis-HCl salt, as described in U.S. 2010/0305125 (Borchardt). In other embodiments, Compound B3 is utilized in the crystalline forms taught by U.S. 2011/0071151 (Zhang et al.). In one embodiment, Compound B3 is ABT-263 free base in a solid crystalline form, as taught by U.S. 2011/0071151 (Zhang et al.). In another embodiment Compound B3 is ABT-263 free base Form I, characterized at least by a powder X-ray diffraction peak at any one or more of the following positions: 6.21, 6.72, 12.17, 18.03 and 20.10° 28, ±0.2° 2Θ, as taught by U.S. 2011/0071151.

In another embodiment, the crystalline form is Form I ABT-263 free base, characterized at least by a powder X-ray diffraction peak at each of the following positions: 6.21, 6.72, 9.66, 10.92, 11.34, 12.17, 14.28, 16.40, 16.95, 17.81, 18.03, 18.47, 19.32, 20.10 and 21.87° 28, ±0.2° 2Θ, as taught by U.S. 2011/0071151.

In another embodiment, the crystalline form is Form II ABT-263 free base, characterized at least by a powder X-ray diffraction peak at any one or more of the following positions: 5.79, 8.60, 12.76, 15.00 and 20.56° 28, ±0.2° 2Θ, as taught by U.S. 2011/0071151.

In a further embodiment, the crystalline form is Form II ABT-263 free base, characterized at least by a powder X-ray diffraction peak at each of the following positions: 5.79, 8.60, 9.34, 10.79, 11.36, 11.59, 12.76, 13.23, 13.73, 14.01, 14.72, 15.00, 16.28, 17.07, 17.48, 18.75, 19.34, 19.71, 20.56 and 21.35° 28, ±0.2° 2Θ, as taught by U.S. 2011/0071151.

In one variation, the Bcl-2 inhibitor is (4-(4-{[2-(4-chlorophenyl)-4,4-dimethylcyclohex-1-en-1-yl]methyl}piperazin-1-yl)-N-({3-nitro-4-[(tetrahydro-2H-pyran-4-yl-methyl)amino]phenyl}sulfonyl)-2-(1H-pyrrolo[2,3-b]pyridin-5-yl-oxy)benzamide), or a pharmaceutically acceptable salt thereof.

In another variation, the Bcl-2 inhibitor is (4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]benzamide, or a pharmaceutically acceptable salt thereof.

In another variation, the Bcl-2 inhibitor is 4-[4-[[2-(4-chlorophenyl)-5,5-dimethyl-1-cyclohexen-1-yl]methyl]-1-piperazinyl]-N-[[4-[[(1R)-3-(4-morpholinyl)-1-[(phenylthio)methyl]propyl]amino]-3 [(trifluoromethyl)sulfonyl]phenyl]sulfonyl]benzamide, or a pharmaceutically acceptable salt thereof.

Additional Bcl-2 inhibitors which may be used in the combinations, methods, and kits herein include those selected from the group of ABT-263, venetoclax (ABT-199), ABT-737, and AT-101 (Gossypol), apogossypol, TW-37, G3139 (Genasense or oblimersen), obatoclax, sabutoclax, HA14-1, antimycin A, and 544563.

The compound names provided herein are named using ChemBioDraw Ultra 12.0. One skilled in the art understands that the compound may be named or identified using various commonly recognized nomenclature systems and symbols. By way of example, the compound may be named or identified with common names, systematic or non-systematic names. The nomenclature systems and symbols that are commonly recognized in the art of chemistry include, for example, Chemical Abstract Service (CAS), ChemBioDraw Ultra, and International Union of Pure and Applied Chemistry (IUPAC).

Also provided herein are isotopically labeled forms of compounds detailed herein. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated, are provided. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of subjects (e.g. humans). Also provided for isotopically labeled compounds described herein are any pharmaceutically acceptable salts, or hydrates, as the case may be.

In some variations, the compounds disclosed herein may be varied such that from 1 to n hydrogens attached to a carbon atom is/are replaced by deuterium, in which n is the number of hydrogens in the molecule. Such compounds may exhibit increased resistance to metabolism and are thus useful for increasing the half life of the compound when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to absorption, distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds provided herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

### Methods of Treatment

The Syk and Bcl-2 inhibitors described herein may be used in a combination therapy. Accordingly, provided herein is a method for treating cancer in a human in need thereof, comprising administering to the human a therapeutically effective amount of a Syk inhibitor and a therapeutically effective amount of a Bcl-2 inhibitor, as described herein.

In some variations, “treatment” or “treating” is an approach for obtaining beneficial or desired results including clinical results. Beneficial or desired clinical results may include one or more of the following:

(i) inhibiting the disease or condition (e.g., decreasing one or more symptoms resulting from the disease or condition, and/or diminishing the extent of the disease or condition);

(ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or

(iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

In some variations, “delaying” the development of a disease or condition means to defer, hinder, slow, retard, stabilize, and/or postpone development of the disease or condition. This delay can be of varying lengths of time, depending on the history of the disease or condition, and/or subject being treated. For example, a method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

### Cancer

In some embodiments, the cancer is carcinoma, sarcoma, melanoma, lymphoma or leukemia. In other embodiments, the cancer is a hematologic malignancy. In some embodiments, the cancer is leukemia (e.g., chronic lymphocytic leukemia), lymphoma (e.g., non-Hodgkin's lymphoma), or multiple myeloma. In other embodiments, the cancer is a solid tumor.

In some variations, the cancer is small lymphocytic lymphoma, non-Hodgkin's lymphoma, indolent non-Hodgkin's lymphoma (iNHL), refractory iNHL, mantle cell lymphoma, follicular lymphoma, lymphoplasmacytic lymphoma, marginal zone lymphoma, immunoblastic large cell lymphoma, lymphoblastic lymphoma, Splenic marginal zone B-cell lymphoma (+/−villous lymphocytes), nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, Mediastinal large B-cell lymphoma, Intravascular large B-cell lymphoma, Primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia.

In other variations, the cancer is pancreatic cancer, urological cancer, bladder cancer, colorectal cancer, colon cancer, breast cancer, prostate cancer, renal cancer, hepatocellular cancer, thyroid cancer, gall bladder cancer, lung cancer (e.g. non-small cell lung cancer, small-cell lung cancer), ovarian cancer, cervical cancer, gastric cancer, endometrial cancer, esophageal cancer, head and neck cancer, melanoma, neuroendocrine cancer, CNS cancer, brain tumors (e.g., glioma, anaplastic oligodendroglioma, adult glioblastoma multiforme, and adult anaplastic astrocytoma), bone cancer, soft tissue sarcoma, retinoblastomas, neuroblastomas, peritoneal effusions, malignant pleural effusions, mesotheliomas, Wilms tumors, trophoblastic neoplasms, hemangiopericytomas, Kaposi's sarcomas, myxoid carcinoma, round cell carcinoma, squamous cell carcinomas, esophageal squamous cell carcinomas, oral carcinomas, cancers of the adrenal cortex, or ACTH-producing tumors.

### Subject

The human in need thereof may be an individual who has or is suspected of having a cancer. In some of variations, the human is at risk of developing a cancer (e.g., a human who is genetically or otherwise predisposed to developing a cancer) and who has or has not been diagnosed with the cancer. As used herein, an “at risk” subject is a subject who is at risk of developing cancer (e.g., a hematologic malignancy). The subject may or may not have detectable disease, and may or may not have displayed detectable disease prior to the treatment methods described herein. An at risk subject may have one or more so-called risk factors, which are measurable parameters that correlate with development of cancer, such as described herein. A subject having one or more of these risk factors has a higher probability of developing cancer than an individual without these risk factor(s).

These risk factors may include, for example, age, sex, race, diet, history of previous disease, presence of precursor disease, genetic (e.g., hereditary) considerations, and environmental exposure. In some embodiments, a human at risk for cancer includes, for example, a human whose relatives have experienced this disease, and those whose risk is determined by analysis of genetic or biochemical markers. Prior history of having cancer may also be a risk factor for instances of cancer recurrence.

In some embodiments, provided herein is a method for treating a human who exhibits one or more symptoms associated with cancer (e.g., a hematologic malignancy). In some embodiments, the human is at an early stage of cancer. In other embodiments, the human is at an advanced stage of cancer.

In some embodiments, provided herein is a method for treating a human who is undergoing one or more standard therapies for treating cancer (e.g., a hematologic malignancy), such as chemotherapy, radiotherapy, immunotherapy, and/or surgery. Thus, in some foregoing embodiments, the combination of a Syk inhibitor and a Bcl-2 inhibitor, as described herein, may be administered before, during, or after administration of chemotherapy, radiotherapy, immunotherapy, and/or surgery.

In another aspect, provided herein is a method for treating a human who is “refractory” to a cancer treatment or who is in “relapse” after treatment for cancer (e.g., a hematologic malignancy). A subject “refractory” to an anti-cancer therapy means they do not respond to the particular treatment, also referred to as resistant. The cancer may be resistant to treatment from the beginning of treatment, or may become resistant during the course of treatment, for example after the treatment has shown some effect on the cancer, but not enough to be considered a remission or partial remission. A subject in “relapse” means that the cancer has returned or the signs and symptoms of cancer have returned after a period of improvement, e.g. after a treatment has shown effective reduction in the cancer, such as after a subject is in remission or partial remission.

In some variations, the human is (i) refractory to at least one anti-cancer therapy, or (ii) in relapse after treatment with at least one anti-cancer therapy, or both (i) and (ii). In some of embodiments, the human is refractory to at least two, at least three, or at least four anti-cancer therapies (including, for example, standard or experimental chemotherapies).

In some embodiments, the subject is a human who has a cancer responsive to Syk activity. In another embodiment, the subject is a human who has a solid cancer tumor which expresses Syk. In some embodiments, the subject is a human who has a 17p deletion, a TP53 mutation, NOTCH1, a SF3B1 mutation, a 11q deletion, or any combination thereof. In one embodiment, the subject is a human who has a 17p deletion, a TP53 mutation, or a combination thereof. In another embodiment, the subject is a human who has NOTCH1, a SF3B1 mutation, a 11q deletion, or any combination thereof.

In another aspect, provided is a method for sensitizing a human who is (i) refractory to at least one chemotherapy treatment, or (ii) in relapse after treatment with chemotherapy, or both (i) and (ii), wherein the method comprises administering a Syk inhibitor in combination with a Bcl-2 inhibitor, as described herein, to the human. A human who is sensitized is a human who is responsive to the treatment involving administration of a Syk inhibitor in combination with a Bcl-2 inhibitor, as described herein, or who has not developed resistance to such treatment.

In another aspect, provided herein is a methods for treating a human for a cancer, with comorbidity, wherein the treatment is also effective in treating the comorbidity. A “comorbidity” to cancer is a disease that occurs at the same time as the cancer.

### Therapeutically Effective Amounts

In some variations, a therapeutically effective amount refers to an amount that is sufficient to effect treatment, as defined below, when administered to a subject (e.g., a human) in need of such treatment. The therapeutically effective amount will vary depending upon the subject and disease condition being treated, the weight and age of the subject, the severity of the disease condition, the manner of administration and the like, which can readily be determined by one of ordinary skill in the art. For example, in one variation, a therapeutically effective amount of Compound A1, or a pharmaceutically acceptable salt or hydrate thereof, is an amount sufficient to modulate Syk expression, and thereby treat a human suffering an indication, or to ameliorate or alleviate the existing symptoms of the indication. In one variation, a therapeutically effective amount of Compound B1, Compound B2 or Compound B3, or a pharmaceutically acceptable salt thereof, is an amount sufficient to modulate activity of anti-apoptotic Bcl-2 proteins, and thereby treat a human suffering an indication, or to ameliorate or alleviate the existing symptoms of the indication.

In another variation, the therapeutically effective amount of the Syk inhibitor, such as Compound A1, or a pharmaceutically acceptable salt or hydrate thereof, may be an amount sufficient to decrease a symptom of a disease or condition responsive to inhibition of Syk activity. In another variation, the therapeutically effective amount of the Bcl-2 inhibitor, such as Compound B1, Compound B2 or Compound B3, or a pharmaceutically acceptable salt thereof, may be an amount sufficient to decrease activity of anti-apoptotic Bcl-2 proteins.

The therapeutically effective amount of the Syk and Bcl-2 inhibitors may also be determined based on data obtained from assays known in the art, including for example, the apoptosis assay described in Example 1 below. In one variation, the therapeutically effective amount of the Syk inhibitor is a dose corresponding to 30 nmol to 700 nmol of the Syk inhibitor used in an apoptosis assay run with 10% serum. In one variation, the therapeutically effective amount of the Bcl-2 inhibitor is a dose corresponding to 1 nmol to 200 nmol of the Bcl-2 inhibitor used in an apoptosis assay run with 10% serum.

In another variation, the Syk inhibitor, such as Compound A1, or a pharmaceutically acceptable salt or hydrate thereof, is administered to the human at a dose resulting in about 50%, about 55%, about 60%, about 65%, about 70%, about 75%, about 80%, about 90%, about 95%, or about 99% Syk target inhibition. In another variation, the Bcl-2 inhibitor, such as Compound B1, Compound B2 or Compound B3, or a pharmaceutically acceptable salt thereof, is administered to the human at a dose resulting in about 50%, about 55%, about 60%, about 65%, about 70%, about 75%, about 80%, about 90%, about 95%, or about 99% Bcl-2 target inhibition.

In some variations, the Syk inhibitor, such as Compound A1, or a pharmaceutically acceptable salt or hydrate thereof, is administered to the human at a dose between 100 mg and 1200 mg, between 100 mg and 800 mg, between 100 mg and 600 mg, between 100 mg and 400 mg, about 100 mg, about 200 mg, about 300 mg, about 400 mg, about 500 mg, about 600 mg, about 700 mg, or about 800 mg.

In some variations, the Bcl-2 inhibitor, such as Compound B1, Compound B2 or Compound B3, or a pharmaceutically acceptable salt thereof, is administered to the human at a daily dose of from about 20 mg to 1,200 mg, from about 20 mg to 1,000 mg, from about 20 mg to 800 mg, from about 20 mg to 500 mg, from about 100 mg to 400 mg, from about 100 mg to 200 mg, about 20 mg, about 40 mg, about 50 mg, about 75 mg, about 100 mg, about 200 mg, about 300 mg, about 400 mg, about 500 mg, about 600 mg, about 700 mg, about 800 mg, about 1,000 mg, or about 1,200 mg.

The therapeutically effective amount of the Syk and Bcl-2 inhibitors may be provided in a single dose or multiple doses to achieve the desired treatment endpoint. As used herein, “dose” refers to the total amount of an active ingredient to be taken each time by a human. The dose administered, for example for oral administration described above, may be administered once daily (QD), twice daily (BID), three times daily, four times daily, or more than four times daily. In some embodiments, the Syk and/or the Bcl-2 inhibitors may be administered once daily. In some embodiments, the Syk and/or the Bcl-2 inhibitors may be administered twice daily.

### Administration

The Syk inhibitor, such as Compound A1, and the Bcl-2 inhibitors, such as Compound B1, Compound B2 and Compound B3, may be administered using any suitable methods known in the art. For example, the compounds may be administered bucally, ophthalmically, orally, osmotically, parenterally (intramuscularly, intraperitoneally intrasternally, intravenously, subcutaneously), rectally, topically, transdermally, or vaginally.

Further, in certain variations, the Syk inhibitor described herein may be administered prior, after or concurrently with the Bcl-2 inhibitors described herein.

### Pharmaceutical Compositions

The Syk and Bcl-2 inhibitors may be administered in the form of pharmaceutical compositions. For example, in some variations, the Syk inhibitor described herein may be present in a pharmaceutical composition comprising the Syk inhibitor, and at least one pharmaceutically acceptable vehicle. In some variations, the Bcl-2 inhibitors described herein may be present in a pharmaceutical composition comprising the Bcl-2 inhibitor, and at least one pharmaceutically acceptable vehicle. Pharmaceutically acceptable vehicles may include pharmaceutically acceptable carriers, adjuvants and/or excipients, and other ingredients can be deemed pharmaceutically acceptable insofar as they are compatible with other ingredients of the formulation and not deleterious to the recipient thereof.

This disclosure therefore provides pharmaceutical compositions that contain the Syk and Bcl-2 inhibitors as described herein, and one or more pharmaceutically acceptable vehicle, such as excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

In some embodiments, the pharmaceutical compositions described herein are formulated in a unit dosage form. The term “unit dosage forms” refers to physically discrete units suitable as unitary dosages for human subjects, each unit containing a predetermined quantity of active material calculated to produce the desired therapeutic effect, in association with a suitable pharmaceutical excipient. Each unit dosage form contains a therapeutically effective amount of the active pharmaceutical agent in question, including those referring to unit dosage forms of Compound A1, Compound B1, Compound B2, or Compound B3, or a pharmaceutically acceptable salt, hydrate, or solvate thereof. In some variations, the pharmaceutical compositions described herein are in the form of a tablet, capsule, or ampoule.

In certain embodiments, the Syk inhibitor described herein, such as Compound A1, or a pharmaceutically acceptable salt or hydrate thereof, is formulated as a tablet. In some variations, such tablet may comprise a mesylate salt of Compound A1, such as a mono-mesylate or a bis-mesylate salt thereof, or a hydrate thereof. Such tablet comprising Compound A1, for example, may be prepared by suitable methods known in the art, such as spray-drying and granulation (e.g., dry granulation).

### Articles of Manufacture and Kits

Compositions (including, for example, formulations and unit dosages) comprising a Syk inhibitor, as described herein, and compositions comprising a Bcl-2 inhibitor, as described herein, can be prepared and placed in an appropriate container, and labeled for treatment of an indicated condition. Accordingly, provided is also an article of manufacture, such as a container comprising a unit dosage form of a Syk inhibitor and a unit dosage form of a Bcl-2 inhibitor, as described herein, and a label containing instructions for use of the compounds. In some embodiments, the article of manufacture is a container comprising (i) a unit dosage form of a Syk inhibitor, as described herein, and one or more pharmaceutically acceptable carriers, adjuvants or excipients; and (ii) a unit dosage form of a Bcl-2 inhibitor, as described herein, and one or more pharmaceutically acceptable carriers, adjuvants or excipients. In one embodiment, the unit dosage form for both the Syk inhibitor and the Bcl-2 inhibitor is a tablet.

Kits also are contemplated. For example, a kit can comprise unit dosage forms of a Syk inhibitor, as described herein, and compositions comprising a Bcl-2 inhibitor, as described herein, and a package insert containing instructions for use of the composition in treatment of a medical condition. In some embodiments, the kits comprises (i) a unit dosage form of the Syk inhibitor, as described herein, and one or more pharmaceutically acceptable carriers, adjuvants or excipients; and (ii) a unit dosage form of a Bcl-2 inhibitor, as described herein, and one or more pharmaceutically acceptable carriers, adjuvants or excipients. In one embodiment, the unit dosage form for both the Syk inhibitor and the Bcl-2 inhibitor is a tablet.

The instructions for use in the kit may be for treating a cancer, including, for example, a hematologic malignancy, as further described herein.

### Combination Therapies

In the present disclosure, in some aspects, the combination therapies and methods described herein concerning the use of Compound A1 with Compound B1, Compound B2, or Compound B3, may be used or further combined with an additional agent or agents selected from the group of a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, an anti-proliferation agent, or any combination thereof.

The combination therapies and methods described herein may be used or combined with an additional one or more of the following additional therapeutic agents: an adenosine A2B receptor (A2B) inhibitor, a BET-bromodomain 4 (BRD4) inhibitor, an isocitrate dehydrogenase 1 (IDH1) inhibitor, an IKK inhibitor, a protein kinase C (PKC) activator or inhibitor, a TPL2 inhibitor, a serine/threonine-protein kinase 1 (TBK1) inhibitor, agents that activate or reactivate latent human immunodeficiency virus (HIV) such as panobinostat or romidepsin, an anti-CD20 antibody such as obinutuzumab, an anti-PD-1 antibody such as nivolimumab (BMS-936558, MDX1106, or MK-34775), and anti-PD-L1 antibodies such as BMS-936559, MPDL3280A, MEDI4736, MSB0010718C, and MDX1105-01.

The combination therapies and methods disclosed herein and the additional one or more therapeutic agents (e.g. an A2B inhibitor, an apoptosis signal-regulating kinase (ASK) inhibitor, a Bruton's tyrosine kinase (BTK) inhibitor, a BRD4 inhibitor, a discoidin domain receptor 1 (DDR1) inhibitor, a histone deacetylase (HDAC) inhibitor, an isocitrate dehydrogenase (IDH) inhibitor, a Janus kinase (JAK) inhibitor, a lysyl oxidase-like protein 2 (LOXL2) inhibitor, a matrix metalloprotease 9 (MMP9) inhibitor, a phosphatidylinositol 3-kinase (PI3K) inhibitor, a PKC activator or inhibitor, a spleen tyrosine kinase (SYK) inhibitor, a TPL2 inhibitor, or a TBK inhibitor) may be further used or combined with a chemotherapeutic agent, an anti-cancer agent, an anti-angiogenic agent, an anti-fibrotic agent, an immunotherapeutic agent, a therapeutic antibody, a radiotherapeutic agent, an anti-neoplastic agent, or any combination thereof.

### Chemotherapeutic Agents

As used herein, the term “chemotherapeutic agent” or “chemotherapeutic” (or “chemotherapy” in the case of treatment with a chemotherapeutic agent) is meant to encompass any non-proteinaceous (i.e., non-peptidic) chemical compound useful in the treatment of cancer.

Chemotherapeutic agents may be categorized by their mechanism of action into, for example, the following groups:
anti-metabolites/anti-cancer agents such as pyrimidine analogs floxuridine, capecitabine, and cytarabine;
purine analogs, folate antagonists, and related inhibitors;
antiproliferative/antimitotic agents including natural products such as vinca alkaloid (vinblastine, vincristine) and microtubule such as taxane (paclitaxel, docetaxel), vinblastin, nocodazole, epothilones, vinorelbine)(NAVELBINE®), and epipodophyllotoxins (etoposide, teniposide);
DNA damaging agents such as actinomycin, amsacrine, busulfan, carboplatin, chlorambucil, cisplatin, cyclophosphamide (CYTOXAN®), dactinomycin, daunorubicin, doxorubicin, epirubicin, iphosphamide, melphalan, merchlorethamine, mitomycin, mitoxantrone, nitrosourea, procarbazine, taxol, taxotere, teniposide, etoposide, and triethylenethiophosphoramide;
antibiotics such as dactinomycin, daunorubicin, doxorubicin, idarubicin, anthracyclines, mitoxantrone, bleomycins, plicamycin (mithramycin), and mitomycin;
enzymes such as L-asparaginase which systemically metabolizes L-asparagine and deprives cells which do not have the capacity to synthesize their own asparagine;
antiplatelet agents;
antiproliferative/antimitotic alkylating agents such as nitrogen mustards cyclophosphamide and analogs (melphalan, chlorambucil, hexamethylmelamine, and thiotepa), alkyl nitrosoureas (carmustine) and analogs, streptozocin, and triazenes (dacarbazine);
antiproliferative/antimitotic antimetabolites such as folic acid analogs (methotrexate);
platinum coordination complexes (cisplatin, oxiloplatinim, and carboplatin), procarbazine, hydroxyurea, mitotane, and aminoglutethimide;
hormones, hormone analogs (estrogen, tamoxifen, goserelin, bicalutamide, and nilutamide), and aromatase inhibitors (letrozole and anastrozole);
anticoagulants such as heparin, synthetic heparin salts, and other inhibitors of thrombin;
fibrinolytic agents such as tissue plasminogen activator, streptokinase, urokinase, aspirin, dipyridamole, ticlopidine, and clopidogrel;
antimigratory agents;
antisecretory agents (breveldin);
immunosuppressives tacrolimus, sirolimus, azathioprine, and mycophenolate;
compounds (TNP-470, genistein) and growth factor inhibitors (vascular endothelial growth factor inhibitors and fibroblast growth factor inhibitors);
angiotensin receptor blockers, nitric oxide donors;
anti-sense oligonucleotides;
antibodies such as trastuzumab and rituximab;
cell cycle inhibitors and differentiation inducers such as tretinoin;
inhibitors, topoisomerase inhibitors (doxorubicin, daunorubicin, dactinomycin, eniposide, epirubicin, etoposide, idarubicin, irinotecan, mitoxantrone, topotecan, and irinotecan), and corticosteroids (cortisone, dexamethasone, hydrocortisone, methylprednisolone, prednisone, and prednisolone);
growth factor signal transduction kinase inhibitors;
dysfunction inducers;
toxins such as Cholera toxin, ricin, Pseudomonas exotoxin, Bordetella pertussis adenylate cyclase toxin, diphtheria toxin, and caspase activators;
and chromatin.

Further examples of chemotherapeutic agents include:
alkylating agents such as thiotepa and cyclophosphamide (CYTOXAN®);
alkyl sulfonates such as busulfan, improsulfan, and piposulfan;
aziridines such as benzodopa, carboquone, meturedopa, and uredopa;
emylerumines and memylamelamines including alfretamine, triemylenemelamine, triethylenephosphoramide, triethylenethiophosphoramide, and trimemylolomelamine;
acetogenins, especially bullatacin and bullatacinone;
a camptothecin, including synthetic analog topotecan;
bryostatin;
callystatin;
CC-1065, including its adozelesin, carzelesin, and bizelesin synthetic analogs;
cryptophycins, particularly cryptophycin 1 and cryptophycin 8;
dolastatin;
duocarmycin, including the synthetic analogs KW-2189 and CBI-TMI;
eleutherobin;
pancratistatin;
a sarcodictyin;
spongistatin;
nitrogen mustards such as chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, and uracil mustard;
nitrosoureas such as carmustine, chlorozotocin, foremustine, lomustine, nimustine, and ranimustine;
antibiotics such as the enediyne antibiotics (e.g., calicheamicin, especially calicheamicin gammall and calicheamicin phiI1), dynemicin including dynemicin A, bisphosphonates such as clodronate, an esperamicin, neocarzinostatin chromophore and related chromoprotein enediyne antibiotic chromomophores, aclacinomycins, actinomycin, authramycin, azaserine, bleomycins, cactinomycin, carabicin, carrninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, and zorubicin;
anti-metabolites such as methotrexate and 5-fluorouracil (5-FU);
folic acid analogs such as demopterin, methotrexate, pteropterin, and trimetrexate;
purine analogs such as fludarabine, 6-mercaptopurine, thiamiprine, and thioguanine;
pyrimidine analogs such as ancitabine, azacitidine, 6-azauridine, carmofur, cytarabine, dideoxyuridine, doxifluridine, enocitabine, and floxuridine;
androgens such as calusterone, dromostanolone propionate, epitiostanol, mepitiostane, and testolactone;
anti-adrenals such as aminoglutethimide, mitotane, and trilostane;
folic acid replinishers such as frolinic acid;
trichothecenes, especially T-2 toxin, verracurin A, roridin A, and anguidine;
taxoids such as paclitaxel (TAXOL) and docetaxel (TAXOTERE®);
platinum analogs such as cisplatin and carboplatin;
aceglatone; aldophosphamide glycoside; aminolevulinic acid; eniluracil; amsacrine; hestrabucil; bisantrene; edatraxate; defofamine; demecolcine; diaziquone; elformthine; elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; polysaccharide-K (PSK); razoxane; rhizoxin; sizofiran; spirogermanium; tenuazonic acid; triaziquone; 2,2′,2″-tricUorotriemylamine; urethane; vindesine; dacarbazine; mannomustine; mitobronitol; mitolactol; pipobroman; gacytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; mitroxantrone; vancristine; vinorelbine (NAVELBINE®); novantrone; teniposide; edatrexate; daunomycin; aminopterin; xeoloda; ibandronate; CPT-11; topoisomerase inhibitor RFS 2000; difluoromethylornithine (DFMO); retinoids such as retinoic acid; capecitabine; FOLFIRI (fluorouracil, leucovorin, and irinotecan);
and pharmaceutically acceptable salts, acids, or derivatives of any of the above.

### Anti-Hormonal Agents

Also included in the definition of “chemotherapeutic agent” are anti-hormonal agents such as anti-estrogens and selective estrogen receptor modulators (SERMs), inhibitors of the enzyme aromatase, anti-androgens, and pharmaceutically acceptable salts, acids or derivatives of any of the above that act to regulate or inhibit hormone action on tumors.

Examples of anti-estrogens and SERMs include, for example, tamoxifen (including NOLVADEX™), raloxifene, droloxifene, 4-hydroxytamoxifen, trioxifene, keoxifene, LY117018, onapristone, and toremifene (FARESTON®).

Inhibitors of the enzyme aromatase regulate estrogen production in the adrenal glands. Examples include 4(5)-imidazoles, aminoglutethimide, megestrol acetate (MEGACE®), exemestane, formestane, fadrozole, vorozole (RIVISOR®), letrozole (FEMARA®), and anastrozole (ARIMIDEX®)

Examples of anti-androgens include flutamide, nilutamide, bicalutamide, leuprohde, and goserelin.

### Anti-Angiogenic Agents

Anti-angiogenic agents include, but are not limited to, retinoid acid and derivatives thereof, 2-methoxyestradiol, ANGIOSTATIN®, ENDOSTATIN®, suramin, squalamine, tissue inhibitor of metalloproteinase-1, tissue inhibitor of metalloproteinase-2, plasminogen activator inhibitor-1, plasminogen activator inbibitor-2, cartilage-derived inhibitor, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism including proline analogs ((1-azetidine-2-carboxylic acid (LACA)), cishydroxyproline, d,I-3,4-dehydroproline, thiaproline, α,α′-dipyridyl, beta-aminopropionitrile fumarate, 4-propyl-5-(4-pyridinyl)-2(3h)-oxazolone, methotrexate, mitoxantrone, heparin, interferons, 2 macroglobulin-serum, chicken inhibitor of metalloproteinase-3 (ChIMP-3), chymostatin, beta-cyclodextrin tetradecasulfate, eponemycin, fumagillin, gold sodium thiomalate, d-penicillamine, beta-1-anticollagenase-serum, alpha-2-antiplasmin, bisantrene, lobenzarit disodium, n-2-carboxyphenyl-4-chloroanthronilic acid disodium or “CCA”, thalidomide, angiostatic steroid, carboxy aminoimidazole, and metalloproteinase inhibitors such as BB-94. Other anti-angiogenesis agents include antibodies, preferably monoclonal antibodies against these angiogenic growth factors: beta-FGF, alpha-FGF, FGF-5, VEGF isoforms, VEGF-C, HGF/SF, and Ang-1/Ang-2.

### Anti-Fibrotic Agents

Anti-fibrotic agents include, but are not limited to, the compounds such as beta-aminoproprionitrile (BAPN), as well as the compounds disclosed in U.S. Pat. No. 4,965,288 relating to inhibitors of lysyl oxidase and their use in the treatment of diseases and conditions associated with the abnormal deposition of collagen and U.S. Pat. No. 4,997,854 relating to compounds which inhibit LOX for the treatment of various pathological fibrotic states, which are herein incorporated by reference. Further exemplary inhibitors are described in U.S. Pat. No. 4,943,593 relating to compounds such as 2-isobutyl-3-fluoro-, chloro-, or bromo-allylamine, U.S. Pat. No. 5,021,456, U.S. Pat. No. 5,059,714, U.S. Pat. No. 5,120,764, U.S. Pat. No. 5,182,297, U.S. Pat. No. 5,252,608 relating to 2-(1-naphthyloxymemyl)-3-fluoroallylamine, and US 2004-0248871, which are herein incorporated by reference.

Exemplary anti-fibrotic agents also include the primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives; semicarbazide and urea derivatives; aminonitriles such as BAPN or 2-nitroethylamine; unsaturated or saturated haloamines such as 2-bromo-ethylamine, 2-chloroethylamine, 2-trifluoroethylamine, 3-bromopropylamine, and p-halobenzylamines; and selenohomocysteine lactone.

Other anti-fibrotic agents are copper chelating agents penetrating or not penetrating the cells. Exemplary compounds include indirect inhibitors which block the aldehyde derivatives originating from the oxidative deamination of the lysyl and hydroxylysyl residues by the lysyl oxidases. Examples include the thiolamines, particularly D-penicillamine, and its analogs such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, and sodium-4-mercaptobutanesulphinate trihydrate.

### Immunotherapeutic Agents

The immunotherapeutic agents include and are not limited to therapeutic antibodies suitable for treating patients. Some examples of therapeutic antibodies include simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, CC49, and 3F8. Rituximab can be used for treating indolent B-cell cancers, including marginal-zone lymphoma, WM, CLL and small lymphocytic lymphoma. A combination of Rituximab and chemotherapy agents is especially effective.

The exemplified therapeutic antibodies may be further labeled or combined with a radioisotope particle such as indium-111, yttrium-90, or iodine-131.

In a certain embodiments, the additional therapeutic agent is a nitrogen mustard alkylating agent. Nonlimiting examples of nitrogen mustard alkylating agents include chlorambucil.

### Lymphoma or Leukemia Combination Therapy

Some chemotherapy agents are suitable for treating lymphoma or leukemia. These agents include aldesleukin, alvocidib, antineoplaston AS2-1, antineoplaston A10, anti-thymocyte globulin, amifostine trihydrate, aminocamptothecin, arsenic trioxide, beta alethine, Bcl-2 family protein inhibitor ABT-263, ABT-199, ABT-737, BMS-345541, bortezomib (VELCADE®), bryostatin 1, busulfan, carboplatin, campath-1H, CC-5103, carmustine, caspofungin acetate, clofarabine, cisplatin, cladribine, chlorambucil, curcumin, cyclosporine, cyclophosphamide, cytarabine, denileukin diftitox, dexamethasone, DT-PACE (dexamethasone, thalidomide, cisplatin, doxorubicin, cyclophosphamide, and etoposide), docetaxel, dolastatin 10, doxorubicin, doxorubicin hydrochloride, enzastaurin, epoetin alfa, etoposide, everolimus (RAD001), fenretinide, filgrastim, melphalan, mesna, flavopiridol, fludarabine, geldanamycin (17-AAG), ifosfamide, irinotecan hydrochloride, ixabepilone, lenalidomide (REVLIMID®, CC-5013), lymphokine-activated killer cells, melphalan, methotrexate, mitoxantrone hydrochloride, motexafin gadolinium, mycophenolate mofetil, nelarabine, oblimersen, obatoclax (GX15-070), oblimersen, octreotide acetate, omega-3 fatty acids, oxaliplatin, paclitaxel, PD0332991, PEGylated liposomal doxorubicin hydrochloride, pegfilgrastim, pentostatin, perifosine, prednisolone, prednisone, R-roscovitine (seliciclib, CYC202), recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, styryl sulphones, tacrolimus, tanespimycin, temsirolimus (CC1-779), thalidomide, therapeutic allogeneic lymphocytes, thiotepa, tipifarnib, bortezomib (VELCADE®, PS-341), vincristine, vincristine sulfate, vinorelbine ditartrate, SAHA (suberanilohydroxamic acid, or suberoyl, anilide, and hydroxamic acid), FR (fludarabine and rituximab), CHOP (cyclophosphamide, doxorubicin, vincristine, and prednisone), CVP (cyclophosphamide, vincristine, and prednisone), FCM (fludarabine, cyclophosphamide, and mitoxantrone), FCR (fludarabine, cyclophosphamide, and rituximab), hyperCVAD (hyperfractionated cyclophosphamide, vincristine, doxorubicin, dexamethasone, methotrexate, and cytarabine), ICE (iphosphamide, carboplatin, and etoposide), MCP (mitoxantrone, chlorambucil, and prednisolone), R-CHOP (rituximab and CHOP), R-CVP (rituximab and CVP), R-FCM (rituximab and FCM), R-ICE (rituximab and ICE), and R-MCP (rituximab and MCP).

One modified approach is radioimmunotherapy, wherein a monoclonal antibody is combined with a radioisotope particle, such as indium-111, yttrium-90, and iodine-131. Examples of combination therapies include, but are not limited to, iodine-131 tositumomab (BEXXAR®), yttrium-90 ibritumomab tiuxetan (ZEVALIN®), and BEXXAR® with CHOP.

The abovementioned therapies can be supplemented or combined with stem cell transplantation or treatment. Therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, low-LET cobalt-60 gamma ray therapy, bleomycin, conventional surgery, radiation therapy, and nonmyeloablative allogeneic hematopoietic stem cell transplantation.

### Non-Hodgkin's Lymphomas Combination Therapy

Treatment of non-Hodgkin's lymphomas (NHL), especially those of B cell origin, includes using monoclonal antibodies, standard chemotherapy approaches (e.g., CHOP, CVP, FCM, MCP, and the like), radioimmunotherapy, and combinations thereof, especially integration of an antibody therapy with chemotherapy.

Examples of unconjugated monoclonal antibodies for the treatment of NHL/B-cell cancers include rituximab, alemtuzumab, human or humanized anti-CD20 antibodies, lumiliximab, anti-TNF-related apoptosis-inducing ligand (anti-TRAIL), bevacizumab, galiximab, epratuzumab, SGN-40, and anti-CD74.

Examples of experimental antibody agents used in treatment of NHL/B-cell cancers include ofatumumab, ha20, PRO131921, alemtuzumab, galiximab, SGN-40, CHIR-12.12, epratuzumab, lumiliximab, apolizumab, milatuzumab, and bevacizumab.

Examples of standard regimens of chemotherapy for NHL/B-cell cancers include CHOP, FCM, CVP, MCP, R-CHOP, R-FCM, R-CVP, and R-MCP.

Examples of radioimmunotherapy for NHL/B-cell cancers include yttrium-90 ibritumomab tiuxetan (ZEVALIN®) and iodine-131 tositumomab (BEXXAR®).

### Mantle Cell Lymphoma Combination Therapy

Therapeutic treatments for mantle cell lymphoma (MCL) include combination chemotherapies such as CHOP, hyperCVAD, and FCM. These regimens can also be supplemented with the monoclonal antibody rituximab to form combination therapies R-CHOP, hyperCVAD-R, and R-FCM. Any of the abovementioned therapies may be combined with stem cell transplantation or ICE in order to treat MCL.

An alternative approach to treating MCL is immunotherapy. One immunotherapy uses monoclonal antibodies like rituximab. Another uses cancer vaccines, such as GTOP-99, which are based on the genetic makeup of an individual patient's tumor.

A modified approach to treat MCL is radioimmunotherapy, wherein a monoclonal antibody is combined with a radioisotope particle, such as iodine-131 tositumomab (BEXXAR®) and yttrium-90 ibritumomab tiuxetan (ZEVALIN®). In another example, BEXXAR® is used in sequential treatment with CHOP.

Other approaches to treating MCL include autologous stem cell transplantation coupled with high-dose chemotherapy, administering proteasome inhibitors such as bortezomib (VELCADE® or PS-341), or administering antiangiogenesis agents such as thalidomide, especially in combination with rituximab.

Another treatment approach is administering drugs that lead to the degradation of Bcl-2 protein and increase cancer cell sensitivity to chemotherapy, such as oblimersen, in combination with other chemotherapeutic agents.

A further treatment approach includes administering mTOR inhibitors, which can lead to inhibition of cell growth and even cell death. Non-limiting examples are temsirolimus (TORISEL®, CCI-779) and temsirolimus in combination with RITUXAN®, VELCADE®, or other chemotherapeutic agents.

Other recent therapies for MCL have been disclosed. Such examples include flavopiridol, PD0332991, R-roscovitine (selicicilib, CYC202), styryl sulphones, obatoclax (GX15-070), TRAIL, Anti-TRAIL death receptors DR4 and DR5 antibodies, temsirolimus (TORISEL®, CC1-779), everolimus (RAD001), BMS-345541, curcumin, SAHA, thalidomide, lenalidomide (REVLIMID®, CC-5013), and geldanamycin (17-AAG).

### Waldenstrom's Macroglobulinemia Combination Therapy

Therapeutic agents used to treat Waldenstrom's Macroglobulinemia (WM) include perifosine, bortezomib (VELCADE®), rituximab, sildenafil citrate (VIAGRA®), CC-5103, thalidomide, epratuzumab (hLL2-anti-CD22 humanized antibody), simvastatin, enzastaurin, campath-1H, dexamethasone, DT-PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium-111 monoclonal antibody MN-14, yttrium-90 humanized epratuzumab, anti-thymocyte globulin, busulfan, cyclosporine, methotrexate, mycophenolate mofetil, therapeutic allogeneic lymphocytes, yttrium-90 ibritumomab tiuxetan, sirolimus, tacrolimus, carboplatin, thiotepa, paclitaxel, aldesleukin, docetaxel, ifosfamide, mesna, recombinant interleukin-11, recombinant interleukin-12, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab, iodine-131 tositumomab, motexafin gadolinium, arsenic trioxide, tipifarnib, autologous human tumor-derived HSPPC-96, veltuzumab, bryostatin 1, PEGylated liposomal doxorubicin hydrochloride, and any combination thereof.

Examples of therapeutic procedures used to treat WM include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme techniques, low-LET cobalt-60 gamma ray therapy, bleomycin, conventional surgery, radiation therapy, and nonmyeloablative allogeneic hematopoietic stem cell transplantation.

### Diffuse Large B-Cell Lymphoma Combination Therapy

Therapeutic agents used to treat diffuse large B-cell lymphoma (DLBCL) include cyclophosphamide, doxorubicin, vincristine, prednisone, anti-CD20 monoclonal antibodies, etoposide, bleomycin, many of the agents listed for WM, and any combination thereof, such as ICE and R-ICE.

### Chronic Lymphocytic Leukemia Combination Therapy

Examples of therapeutic agents used to treat chronic lymphocytic leukemia (CLL) include chlorambucil, cyclophosphamide, fludarabine, pentostatin, cladribine, doxorubicin, vincristine, prednisone, prednisolone, alemtuzumab, many of the agents listed for WM, and combination chemotherapy and chemoimmunotherapy, including the following common combination regimens: CVP, R-CVP, ICE, R-ICE, FCR, and FR.

### Myelofibrosis Combination Therapy

Myelofibrosis inhibiting agents include, but are not limited to, hedgehog inhibitors, histone deacetylase (HDAC) inhibitors, and tyrosine kinase inhibitors. A non-limiting example of hedgehog inhibitors is saridegib.

Examples of HDAC inhibitors include, but are not limited to, pracinostat and panobinostat.

A non-limiting example of a tyrosine kinase inhibitor is lestaurtinib.

### Kinase Inhibitors

In one embodiment, the compound described herein may be used or combined with one or more additional therapeutic agents. The one or more therapeutic agents include, but are not limited to, an inhibitor of Abl, activated CDC kinase (ACK), adenosine A2B receptor (A2B), apoptosis signal-regulating kinase (ASK), Auroa kinase, Bruton's tyrosine kinase (BTK), BET-bromodomain (BRD) such as BRD4, c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptors (EGFR), focal adhesion kinase (FAK), Flt-3, FYN, glycogen synthase kinase (GSK), HCK, histone deacetylase (HDAC), IKK such as IKKβε, isocitrate dehydrogenase (IDH) such as IDH1, Janus kinase (JAK), KDR, lymphocyte-specific protein tyrosine kinase (LCK), lysyl oxidase protein, lysyl oxidase-like protein (LOXL), LYN, matrix metalloprotease (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK) such as protein kinase A, B, and/or C, PYK, spleen tyrosine kinase (SYK), serine/threonine kinase TPL2, serine/threonine kinase STK, signal transduction and transcription (STAT), SRC, serine/threonine-protein kinase (TBK) such as TBK1, TIE, tyrosine kinase (TK), vascular endothelial growth factor receptor (VEGFR), YES, or any combination thereof.

### Apoptosis Signal-Regulating Kinase (ASK) Inhibitors

ASK inhibitors include ASK1 inhibitors. Examples of ASK1 inhibitors include, but are not limited to, those described in WO 2011/008709 (Gilead Sciences) and WO 2013/112741 (Gilead Sciences).

### Bruton's Tyrosine Kinase (BTK) Inhibitors

Examples of BTK inhibitors include, but are not limited to, ibrutinib, HM71224, GS-4059 (ONO-4059), and CC-292.

### Discoidin Domain Receptor (DDR) Inhibitors

DDR inhibitors include inhibitors of DDR1 and/or DDR2. Examples of DDR inhibitors include, but are not limited to, those disclosed in WO 2014/047624 (Gilead Sciences), US 2009-0142345 (Takeda Pharmaceutical), US 2011-0287011 (Oncomed Pharmaceuticals), WO 2013/027802 (Chugai Pharmaceutical), and WO 2013/034933 (Imperial Innovations).

### Histone Deacetylase (HDAC) Inhibitors

Examples of HDAC inhibitors include, but are not limited to, pracinostat and panobinostat.

### Janus Kinase (JAK) Inhibitors

JAK inhibitors inhibit JAK1, JAK2, and/or JAK3. Examples of JAK inhibitors include, but are not limited to, Compound A, ruxolitinib, fedratinib, tofacitinib, baricitinib, lestaurtinib, pacritinib, XL019, AZD1480, INCB039110, LY2784544, BMS911543, and NS018.

### Lysyl Oxidase-Like Protein (LOXL) Inhibitors

LOXL inhibitors include inhibitors of LOXL1, LOXL2, LOXL3, LOXL4, and/or LOXL5. Examples of LOXL inhibitors include, but are not limited to, the antibodies described in WO 2009/017833 (Arresto Biosciences).

Examples of LOXL2 inhibitors include, but are not limited to, the antibodies described in WO 2009/017833 (Arresto Biosciences), WO 2009/035791 (Arresto Biosciences), and WO 2011/097513 (Gilead Biologics).

### Matrix Metalloprotease (MMP) Inhibitors

MMP inhibitors include inhibitors of MMP1 through 10. Examples of MMP9 inhibitors include, but are not limited to, marimastat (BB-2516), cipemastat (Ro 32-3555), and those described in WO 2012/027721 (Gilead Biologics).

### Phosphatidylinositol 3-Kinase (PI3K) Inhibitors

PI3K inhibitors include inhibitors of PI3Kγ, PI3Kδ, PI3Kβ, PI3Kα, and/or pan-PI3K. Examples of PI3K inhibitors include, but are not limited to, wortmannin, BKM120, CH5132799, XL756, and GDC-0980.

Examples of PI3Kγ inhibitors include, but are not limited to, ZSTK474, AS252424, LY294002, and TG100115.

Examples of PI3Kδ inhibitors include, but are not limited to, Compound B, Compound C, Compound D, Compound E, PI3K II, TGR-1202, AMG-319, GSK2269557, X-339, X-414, RP5090, KAR4141, XL499, OXY111A, IPI-145, IPI-443, and the compounds described in WO 2005/113556 (ICOS), WO 2013/052699 (Gilead Calistoga), WO 2013/116562 (Gilead Calistoga), WO 2014/100765 (Gilead Calistoga), WO 2014/100767 (Gilead Calistoga), and WO 2014/201409 (Gilead Sciences).

Examples of PI3Kβ inhibitors include, but are not limited to, GSK2636771, BAY 10824391, and TGX221.

Examples of PI3Kα inhibitors include, but are not limited to, buparlisib, BAY 80-6946, BYL719, PX-866, RG7604, MLN1117, WX-037, AEZA-129, and PA799.

Examples of pan-PI3K inhibitors include, but are not limited to, LY294002, BEZ235, XL147 (SAR245408), and GDC-0941.

### Spleen Tyrosine Kinase (SYK) Inhibitors

Examples of SYK inhibitors include, but are not limited to, tamatinib (R406), fostamatinib (R788), PRT062607, BAY-61-3606, NVP-QAB 205 AA, R112, R343, and those described in U.S. Pat. No. 8,450,321 (Gilead Connecticut).

### Tyrosine-Kinase Inhibitors (TKIs)

TKIs may target epidermal growth factor receptors (EGFRs) and receptors for fibroblast growth factor (FGF), platelet-derived growth factor (PDGF), and vascular endothelial growth factor (VEGF). Examples of TKIs that target EGFR include, but are not limited to, gefitinib and erlotinib. Sunitinib is a non-limiting example of a TKI that targets receptors for FGF, PDGF, and VEGF.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example 1

### Human CLL Apoptosis Assay

Peripheral blood mononuclear cells (PBMCs) were isolated from primary chronic lymphocytic leukemia (CLL) patients and cultured for 3-5 hours in Lymphocyte Growth Medium (LGM, RPMI 1640, 1 mM Sodium Pyruvate, 10 mM HEPES, pH 7.4, 100 U/mL Penicillin/100 μg/mL Streptomycin, 55 μM β-Mercaptoethanl, 2 mM GlutaMAX, and 10% FBS) at 37° C. with 5% CO₂. Cells were then centrifuged at room temperature for 10 minutes and resuspended in an appropriate volume of LGM for plating (maximum cell density=3.12×10⁶/ml). Final assay wells were set up in U-bottom 96-well tissue culture plates with HS-5 co-culture (plates were coated with 3×10⁴ HS-5 cells in 100 μl overnight at 37° C. prior to the assay) or no co-culture.

Cell suspensions (80 μL, 2.5×10⁵-9.4×10⁴) were added to the plate and incubated for 1 hour prior to stimulation with αIgM/αIgG (7.8 μg/well) and αCD40 (4 μg/well). Cells were incubated with compounds for about 66 hours at 37° C. After incubation, the cells were transferred to deeper plates and washed once with 500 μL of 1×PBS^{+/+}. Cells were resuspended in Invitrogen's aqua Live/Dead reagent according to manufacturer's directions and incubated 30 minutes on ice. Aqua Live/Dead was quenched with an equal volume of PBS^{+/+} with 4% FBS (FACS buffer). Cells were centrifuged and labeled with αCD5⁻PE, αCD19-BV421 and AnnexinV-APC in a total volume of 85 and incubated 30 minutes on ice. After labeling, cells were rinsed twice in FACS buffer and then fixed with BD Fixation buffer for 30 minutes on ice. Cells were rinsed twice with FACS buffer and analyzed.

For the apoptosis analysis, flow cytometric sampling of 5000-20,000 total events were collected on a BD FACS Canto II instrument using a high throughput screen (HTS) autosampler for analysis of apoptosis. CD5⁺/CD19⁺ cells were identified and subsequently gated for AnnexinV⁺/LiveDead and AnnexinV⁺/LiveDead⁺ populations.

Flow cytometric data were extracted to a flow cytometry standard (fcs) file. Average percentages of AnnexinV⁺ cells were determined for the positive control and negative wells (no compound). The percentage of AnnexinV⁺ cells represented the percentage or levels of apoptosis. The results of CLL cells without HS-5 co-culture are summarized in Table 1. Similar results were obtained for CLL cells with HS-5 co-culture.

TABLE 1
Percentage of the AnnexinV⁺ cells from CLL patients treated with Compound
A1 and Compound B1

| | Sample | | | | | | | | | | | |
|---|---|---|---|---|---|---|---|---|---|---|---|---|
| | 1 | 1 | 1 | 1 | 2 | 2 | 2 | 2 | 3 | 3 | 3 | 3 |
| Compound A1 (nM) | 0 | 300 | 100 | 30 | 0 | 300 | 100 | 30 | 0 | 300 | 100 | 30 |
| 200 nM Compound B1 | 46 | 76 | 58 | 52 | 97 | 98 | 98 | 98 | 100 | 100 | 100 | 100 |
| 100 nM Compound B1 | 36 | 54 | 30 | 36 | 95 | 98 | 97 | 97 | 99 | 100 | 100 | 100 |
| 50 nM Compound B1 | 31 | 42 | 31 | 30 | 93 | 97 | 96 | 96 | 97 | 99 | 99 | 98 |
| 25 nM Compound B1 | 24 | 34 | 24 | 22 | 89 | 94 | 92 | 91 | 89 | 96 | 94 | 90 |
| 12.5 nM Compound B1 | 19 | 28 | 20 | 17 | 76 | 89 | 85 | 85 | 69 | 80 | 74 | 68 |
| 6.3 nM Compound B1 | 16 | 23 | 16 | 16 | 55 | 79 | 72 | 66 | 50 | 63 | 57 | 48 |
| 3.1 nM Compound B1 | 15 | 19 | 14 | 14 | 35 | 66 | 56 | 43 | 33 | 46 | 39 | 32 |
| 1.6 nM Compound B1 | 15 | 18 | 14 | 12 | 18 | 51 | 37 | 27 | 21 | 32 | 28 | 20 |
| 0.8 nM Compound B1 | 13 | 16 | 13 | 13 | 10 | 38 | 21 | 15 | 16 | 26 | 20 | 16 |
| 0 nM Compound B1 | NA | 16 | 12 | 12 | NA | 20 | 12 | 9 | NA | 18 | 15 | 13 |
| Stimulated | 13 | 13 | 13 | 13 | 6 | 6 | 6 | 6 | 12 | 12 | 12 | 12 |
| Unstimulated | 67 | 67 | 67 | 67 | 25 | 25 | 25 | 25 | 13 | 13 | 13 | 13 |

What is claimed is:

1. A method for treating cancer in a human in need thereof, comprising administering to the human a therapeutically effective amount of a Syk inhibitor and a therapeutically effective amount of a Bcl-2 inhibitor, wherein:
the Syk inhibitor is a compound of formula A1: 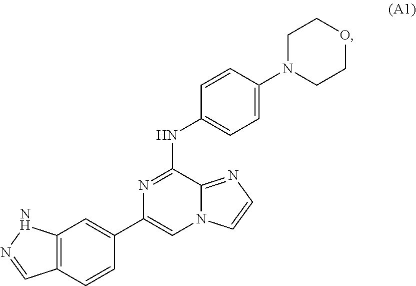 [SMILES: c1cn2cc(-c3ccc4cn[nH]c4c3)nc(Nc3ccc(N4CCOCC4)cc3)c2n1]
or a pharmaceutically acceptable salt or hydrate thereof; and
the Bcl-2 inhibitor is selected from the group a compound of Formula B1, a compound of Formula B2, and a compound of Formula B3: 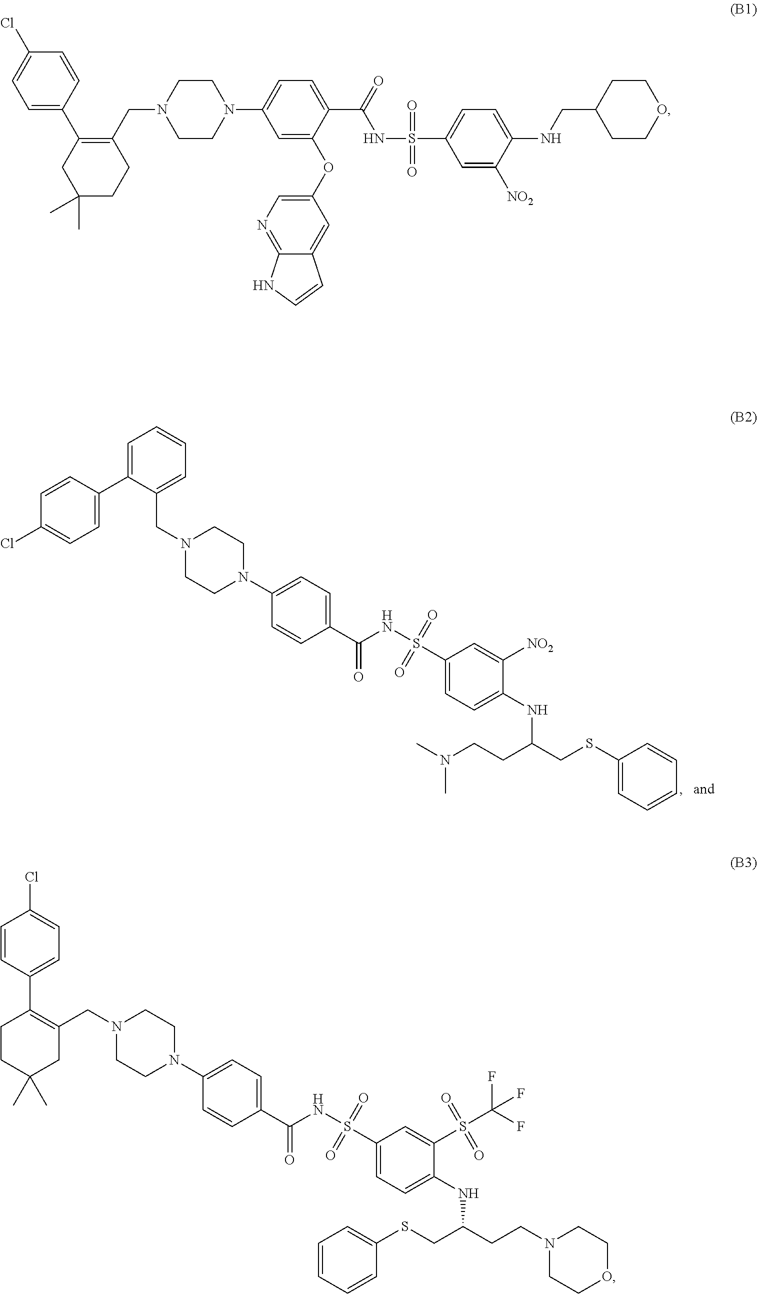 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1 ; CN(C)CCC(CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]
or a pharmaceutically acceptable salt thereof.

2. The method of claim 1, wherein the pharmaceutically acceptable salt of the Syk inhibitor is a mesylate salt, or a hydrate thereof.

3. The method of claim 2, wherein the mesylate salt is a mono-mesylate salt or a bis-mesylate salt, or a combination thereof.

4. The method of claim 1, wherein the Bcl-2 inhibitor is a compound of formula B1: 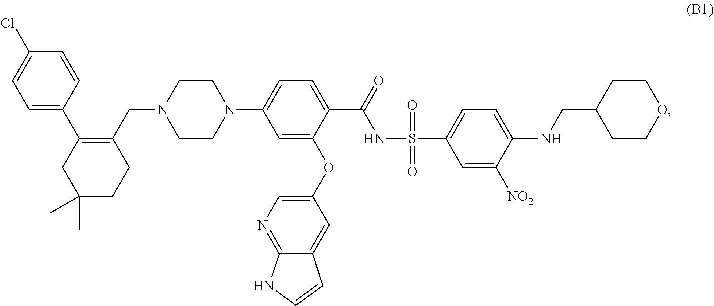 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]
or a pharmaceutically acceptable salt thereof.

5. The method of claim 4 wherein the compound of Formula B1 is of a form selected from the group of a free base anhydrate, a free base dichloromethane solvate, a free base ethyl acetate solvate, a free base acetonitrile solvate, a free base acetone solvate, a hydrochloride, a hydrochloride hydrate, a free base sulfate, and a free base tetrahydrofuran.

6. The method of claim 1, wherein the Bcl-2 inhibitor is a compound of formula B2: 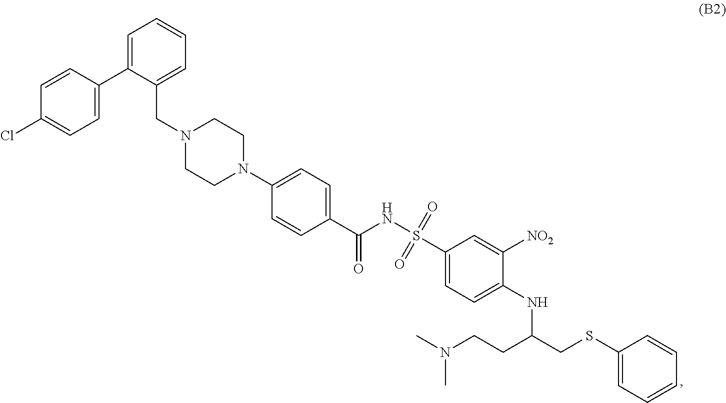 [SMILES: CN(C)CCC(CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]
or a pharmaceutically acceptable salt thereof.

7. The method of claim 1, wherein the Bcl-2 inhibitor is a compound of formula B3: 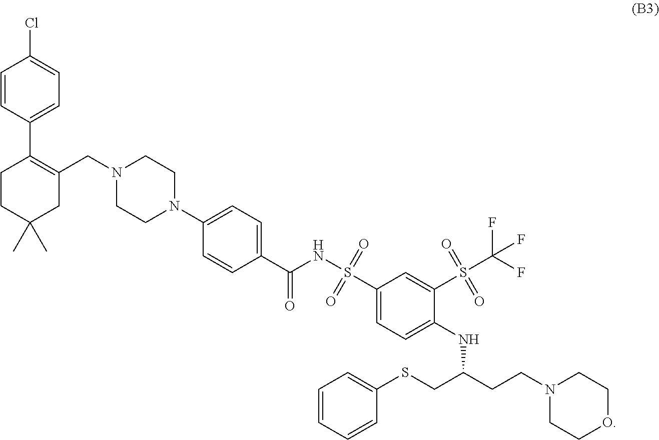 [SMILES: CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]
or a pharmaceutically acceptable salt thereof.

8. The method of claim 7 wherein the Bcl-2 inhibitor is a bis-HCl salt of the compound of Formula B3.

9. The method of claim 1, wherein the Syk inhibitor is administered intravenously, intramuscularly, parenterally, nasally or orally.

10. The method of claim 1, wherein the Bcl-2 inhibitor is administered intravenously, intramuscularly, parenterally, nasally or orally.

11. The method of claim 1, wherein the Syk inhibitor is administered prior, after or concurrently with the Bcl-2 inhibitor.

12. The method of claim 1, wherein the cancer is a hematologic malignancy.

13. The method of claim 1, wherein the cancer is leukemia, lymphoma, or multiple myeloma.

14. The method of claim 1, wherein the cancer is small lymphocytic lymphoma, non-Hodgkin's lymphoma, indolent non-Hodgkin's lymphoma, refractory iNHL, mantle cell lymphoma, follicular lymphoma, lymphoplasmacytic lymphoma, marginal zone lymphoma, immunoblastic large cell lymphoma, lymphoblastic lymphoma, Splenic marginal zone B-cell lymphoma (+/−villous lymphocytes), Nodal marginal zone lymphoma (+/−monocytoid B-cells), extranodal marginal zone B-cell lymphoma of mucosa-associated lymphoid tissue type, cutaneous T-cell lymphoma, extranodal T-cell lymphoma, anaplastic large cell lymphoma, angioimmunoblastic T-cell lymphoma, mycosis fungoides, B-cell lymphoma, diffuse large B-cell lymphoma, Mediastinal large B-cell lymphoma, Intravascular large B-cell lymphoma, Primary effusion lymphoma, small non-cleaved cell lymphoma, Burkitt's lymphoma, multiple myeloma, plasmacytoma, acute lymphocytic leukemia, T-cell acute lymphoblastic leukemia, B-cell acute lymphoblastic leukemia, B-cell prolymphocytic leukemia, acute myeloid leukemia, chronic lymphocytic leukemia, juvenile myelomonocytic leukemia, minimal residual disease, hairy cell leukemia, primary myelofibrosis, secondary myelofibrosis, chronic myeloid leukemia, myelodysplastic syndrome, myeloproliferative disease, or Waldestrom's macroglobulinemia.

15. The method of claim 1 in which the cancer is chronic lymphocytic leukemia.

16. The method of claim 1 in which the cancer is acute lymphocytic leukemia.

17. The method of claim 1 in which the cancer is non-Hodgkin's lymphoma.

18. The method of claim 1 wherein the human is (i) refractory to at least one anti-cancer therapy, or (ii) in relapse after treatment with at least one anti-cancer therapy, or both refractory to at least one anti-cancer therapy and in relapse after treatment with at least one anti-cancer therapy.

19. A kit comprising:
(i) a pharmaceutical composition comprising a therapeutically effective amount of a Syk inhibitor, wherein the Syk inhibitor is a compound of Formula A1: 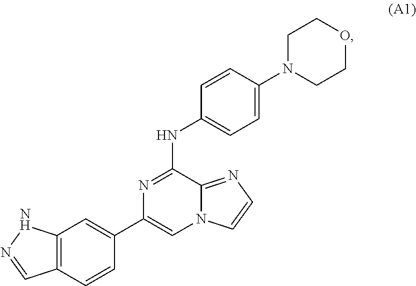 [SMILES: c1cn2cc(-c3ccc4cn[nH]c4c3)nc(Nc3ccc(N4CCOCC4)cc3)c2n1]
or a pharmaceutically acceptable salt or hydrate thereof;
(ii) a pharmaceutical composition comprising a therapeutically effective amount of a Bcl-2 inhibitor, wherein the Bcl-2 inhibitor is selected from the group of a compound of Formula B1, a compound of Formula B2, and a compound of Formula B3: 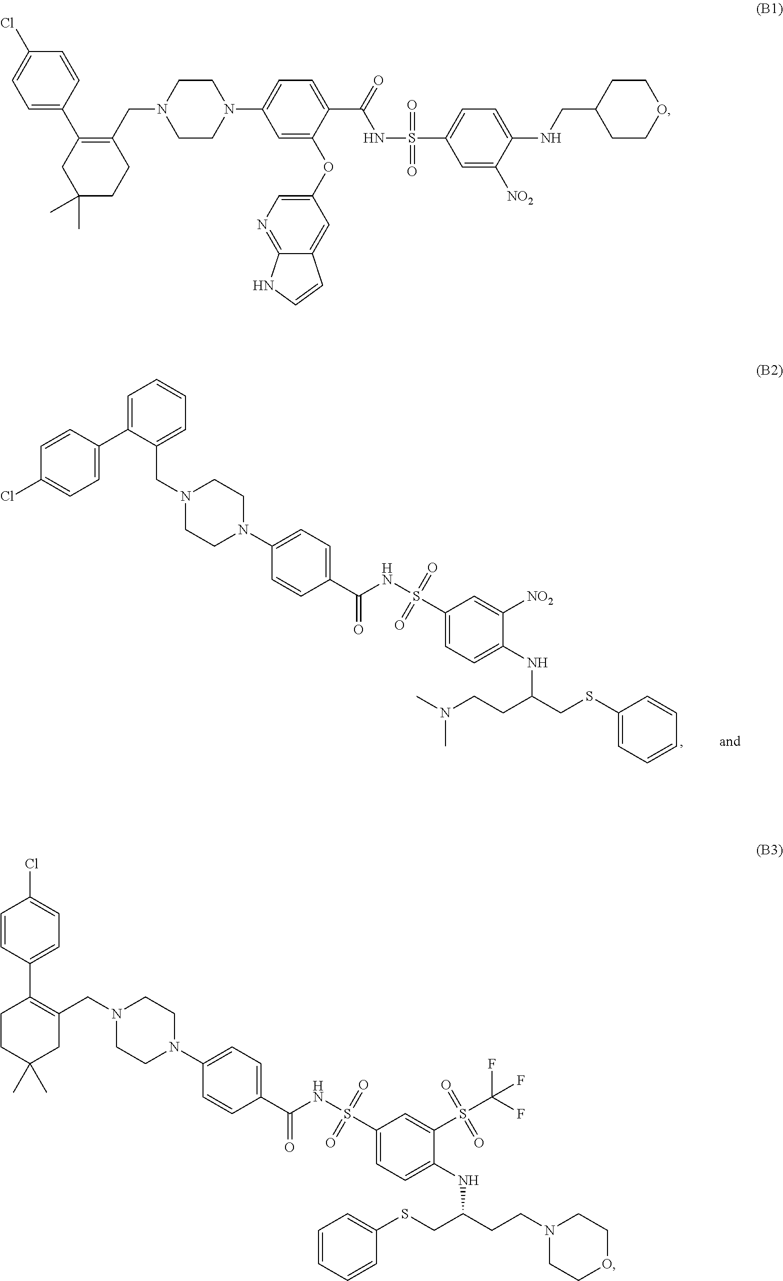 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1 ; CN(C)CCC(CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-] ; CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]
or a pharmaceutically acceptable salt thereof.

20. The kit of claim 12, further comprising a package insert containing instructions for use of the pharmaceutical compositions in treating a cancer.
